(19) United States
(12) Patent Application Publication
(10) Pub. No.: US 2005/0254319 A1
(43) Pub. Date: Nov. 17, 2005

(54) Polymer bound donor-acceptor-donor compounds and their use in a 3-dimensional optical memory

(75) Inventors: Ortal Alpert, Jerusalem (IL); Nissim Garti, Jerusalem (IL); Elena Panitkova, Jerusalem (IL); Andrew N Shipway, Jerusalem (IL); Thierry Wasserman, Fel Aviv (IL)

Correspondence Address: BROWDY AND NEIMARK, P.L.L.C.;624 NINTH STREET, NW, SUITE 300, WASHINGTON, DC, 20001-5303, US

(73) Assignee: MEMPILE INC., Wilmington, DE (US)

(21) Appl. No.: 10/505,109

(22) PCT Filed: Feb. 20, 2003
(86) PCT No.: PCT/IL03/00136

§ 371 Date: May 25, 2005

(30) Foreign Application Priority Data
Feb. 21, 2002 (IL) 148310
Feb. 21, 2002 (IL) 148312
Mar. 14, 2002 (IL) 148707

(51) Int. Cl. G11C 7/00

(52) U.S. Cl. 365/200

(57) ABSTRACT

The present invention is directed to a 3-dimensional optical memory comprising as an active medium a compound capable of interconverting from one isomeric form to another by interaction of light. Said compound is bound to a polymer for achieving a uniform memory unit.

### FIELD OF THE INVENTION

This invention relates to polymer bound compounds, to compounds, processes for their preparation, and a 3-dimensional optical data storage and retrieval system comprising such compounds.

### BACKGROUND OF THE INVENTION

The following publications are referred to in the present description:
1) U.S. Pat. No. 5,592,462;
2) U.S. Pat. No. 5,268,862; and
3) WO 01/73,779.

The computerized era has raised the need to provide reliable means for the storage of large amounts of data. Ever-growing amounts of data are generated nowadays in personal and commercial computers, and with the progress of technology, this demand will surely grow. One approach to address this need is to use optical methods for the storage of data, allowing the stored information to be maintained undamaged for long periods of time, with no apparent loss of information. Three-dimensional data storage offers the possibility of holding terabytes of data on media similar in size to today's optical media (CD, DVD). In order to access the data points in the media, however, 3D addressing is required. This can be achieved by one light beam or by the interaction of two or more light beams in the substance. As an example, two focused, crossing laser beams are able to define a specific point. In order to write data to the 3D media, there needs to be a chemical species within the media that is able to adopt two different forms. Furthermore, this species must be switchable between the two forms by the multiple light interaction, and not by any of the light beams independently. In the past, such devices have been developed based on two-photon absorption by known photoisomerizable molecules. These molecules have low two-photon cross-sections, so relatively high-powered light sources are required, leading to expensive devices, slow data access, and danger of damage to the media.

U.S. Pat. No. 5,592,462 (Beldock) describes a three dimensional system for optical data storage and retrieval. According to this publication, incorporated herein as a reference, the data is stored and retrieved by irradiating the storage medium with two interfering light beams. The use of two light beams allows the definition of a particular portion of the volume being written or read at every instance.

U.S. Pat. No. 5,268,862 (Rentzepis) describes an active medium for use in a system of the kind describe by Beldock. The medium makes use of two forms of a spirobenzopyran derivative to represent the two binary states. However, the memory is maintained at a temperature lower than room temperature, typically at −78 C. Thus writing, storing the written information, and reading are preformed at this low temperature. Raising the temperature erases the entire stored information, as one of the states is stable at room temperature for only 150 seconds. The maintenance of such a memory is expensive and cannot be used commercially.

WO 01/73,779 describes the use of stilbene diethanol and substituted and non-substituted stilbene diethylacetate in a 3-D memory.

### SUMMARY OF THE INVENTION

The present invention is based on the fact that active compounds that may be used as the active medium for a 3-dimensional memory are bound to a polymer in order to achieve a structured, ordered memory. Thus the present invention provides new polymer-bound compounds, new compounds, methods for their synthesis and their use in 3-D memory. The polymer-bound compounds of the present invention are of the general formula (I): 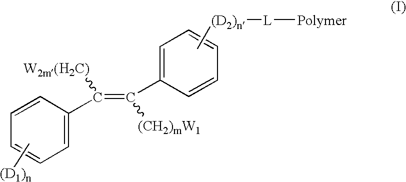
wherein the orientation of the substituents is either cis or trans and wherein m and m′ are independently 0, 1 or 2; n and n′ are independently 0, 1, 2, or 3; W₁ and W₂ are independently selected from CN, OH, C≡CR, COOH, COOR wherein R is a straight or branched C₁₋₄-allyl group, CONH₂, OCH₂OCH₃. D₁ and D₂ are independently selected from R. NO₂, halogen or O—R wherein R is a hydrogen, C₁₋₄-alkyl group optionally substituted by halogen. L is a linking group selected from (CH₂)ₐX or O(CH₂)_{b}X, (OCH₂CH₂)ₙ a and b being 0-10, n being 1-4 and X being O—C(═O)C—.

The polymer is chosen from poly(alkylacrylate)s or their copolymers such as a copolymer with stryrene. More specifically the polymer is poly(methyl methacrylate).

The invention is further directed to a process for the synthesis of compounds of formula (I). The synthesis comprises of derivatizing a compound of formula (II) to a compound of formula (III) 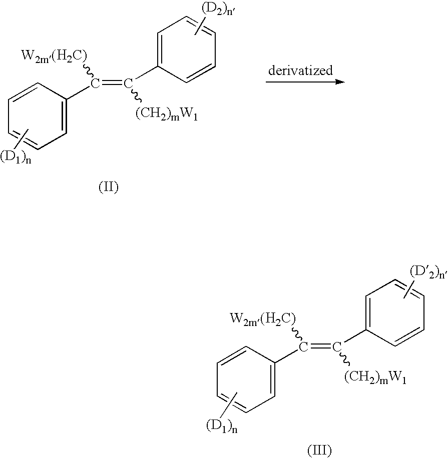
wherein the orientation of the substituents is either cis or trans and wherein n, n′, m, m′, W₁, W₂, D₁, D₂ are as defined above and D₂′ is a derivative of a D group as defined above, e.g. OH, OR or CH₂X, X being a halogen or COOR, R being a C₁-C₄-algyl group. In the next step the compound of formula (H) is reacted with a bi-functional spacer selected from X(CH₂)ₐX or O(CH₂)_{b}X, (OCH₂CH₂)ₙX a and b being 0-10, n being 14 and X being a functional group capable of attaching by chemical means to the polymer or a polymerizable group, e.g. OH, O—C(═O)C═CH₂, halogen which forms the linking moiety L to the polymer (after interacting at both ends) as defined above.

Alternatively, a compound of formula (II) may be derivitized and functionalized with the bi-fucntional spacer to form a compound that is capable of being subsequently polymerized in the presence of an appropriate monomer to yield a copolymer.

The invention is yet further directed to compounds of formula (II) and (III) being novel compounds and to their synthesis.

In the synthesis of compounds of formula (II), for the case wherein W₁ or W₂ are COOH, COOR, OCH₂OCH₃ or for the case wherein m or m′=2 and W₁ or W₂ is OH the process comprises reacting a substituted or non-substituted benzil in a Reformatsky reaction to obtain an intermediate which is further reacted by a McMurry reaction to give a compound of formula (H) with m, m′, W₁ and W₂ as defined above. The resulting compound may further be chemically modified.

For the case wherein W₁ or W₂ are CN and m′=0, substituted or non-substituted phenylacetonitrile is coupled then modified if necessary, to yield the required compound of formula (II).

Compounds of formula (II) wherein m=m′=1 or 2 and W₁ and W₂ are CN may be obtained from the corresponding compound wherein W₁ and W₂ are OH wherein the di-alcohol stilbene is further reacted to yield the di-nitrile compound.

Several of the compounds of formula (II) are conjugated Donor-Acceptor-Donor structures, where the W_{2m′(CH}₂)C═(CH₂)ₘW₁ moiety is an Acceptor moiety which is “sandwiched” between the two substituted phenyl rings which are Donor moieties. Thus the invention is also further directed to the use of conjugated Donor-Acceptor-Donor compounds of the present invention (compounds of formula II) in a 3-D memory such as described in WO 01/73,779 wherein the active medium comprises compounds of formula (II) bound to a polymer in order to achieve an ordered memory.

### BRIEF DESCRIPTION OF THE DRAWINGS

In order to understand the invention and to see how it may be carried out in is practice, a preferred embodiment will now be described, by way of non-limiting example only, with reference to the accompanying drawings, in which:

FIG. 1 displays the chemical formulae of several donor-acceptor-donor compounds, which may be used in a 3-D memory according to the invention.

FIG. 2 shows an ultraviolet-visible spectrum, “write” region of the compound 4,4′-dimethyl-(α,α-dicyanostilbene.

FIG. 3 shows an infrared spectrum, “read” region of a compound of the compound 4,4′-dimethyl-α,α-dicyanostilbene.

FIGS. 4A and 4B show thermodynamic stability studies measured by (A) ultraviolet spectrum, by (B) NMR for the two cis and trans states of the compound 4,4′-dimethoxy-(α,α-dicyanostilbene.

FIG. 5 shows an infrared spectrum of a polymer-bound α,α-dicyanostilbene through a spacer.

FIG. 6 shows an ultraviolet spectrum of a copolymer made of α,α-dicyanostilbene converted to a monomer and subsequently polymerized in the presence of methylmethacrylate.

FIG. 7 shows the Nuclear Magnetic Resonance spectrum of a compound used as the active chromophore used as a monomer to be polymerized.

FIG. 8 (A) shows the Nuclear Magnetic Resonance spectrum of 4,4′-dimethyl-α,α-dicyanostilbene bound through diethylene glycol as a spacer to PMMA in its trans geometry and (13) the ultraviolet spectrum of a the compound shown in (A) in the logical ‘0’ and ‘1’ steps.

FIG. 9 shows a 3-dimensional memory unit of the present invention composed of 4-methoxystilbene-α,α-dicyanide bound to a polymer through a spacer.

### DETAILED DESCRIPTION OF THE INVENTION

As mentioned above the present invention deals with compounds bound to a polymer (compounds of formula (I)), a process for their preparation and their use in 3-dimensional memory such as described in WO 01/73,779 wherein the compounds of the present invention form an active medium suitable for storing and retrieving data. Preferably, the compounds bound to the polymer are donor-acceptor-donor compounds, hence the active medium of the 3-dimensional memory is comprised of donor-acceptor-donor compounds of formula (II). The compound of formula (II) of the present invention are part of an active medium suitable for storing and retrieving data. The basis of the 3-dimensional memory is the interaction of the compounds with incident light to interconvert the active compounds from one chemical structure to a different chemical structure. The active compounds may be regarded as chromophores. The development of viable 3D optical data storage requires a photoisomerizable species that has a high multi-photon cross-section. Simple molecules with this property have been designed for nonlinear optical applications by the application of a conjugated donor-acceptor-donor structure (DAD). In this paradigm, a long conjugated molecule carries charge-transfer donors at its ends and a charge-transfer acceptor in its middle section. The longer the molecule, and the stronger the donors and acceptors, the better the multiphoton absorbance characteristics are. In addition, other similar architectures, e.g. acceptor-donor-acceptor, achieve similar results. Examples of donor functionalization can include: ethers and thioethers, alcohols, thiols and their salts, amines, biphenyls, heteroaromatics e.g. tetrathiafulvalene, alkyl. Examples of acceptor functionalization can include: pyridinium and ammonium salts, multiple bonds, azobenzenes, nitrites, halides, nitro compounds. More complex conjugated systems may also be used as donor or acceptor groups.

In a 3D memory each chemical structure represents a different mode, such as for illustration, ‘0’ and ‘1’ in a binary representation. The different chemical structures may be two separate geometric forms, i.e. cis and trans. An active medium should thus be understood as a plurality of molecules bound to a polymer confined within a given volume or a plurality of molecules (II) that form part of the is polymer that are capable of changing their states from one isomeric form to another upon an interaction with light. The first excitation energy corresponds to the energy required to photochemically convert a molecule of the active medium from the first chemical form to a second one. According to a preferred embodiment of the present invention, the memory apparatus according to the invention comprises: means for directing light beam having a first energy, less than that of the first excitation energy to a selected portion of the active medium, and means for directing additional light beams having additional energies different from the first threshold energy, to the same selected portion of the active medium. The combined energy of the first light beam and the additional light beams are substantially equal to the first excitation energy. A system suitable for this embodiment is described in ref. 2, and in ref 1, for the case wherein one additional light beam is used. In a preferred embodiment of the invention, the isomeric forms of the active medium have a substantially different interactions with energy of a second excitation energy, thus allowing the retrieval of the information in a manner similar to its preferred manner of writing, described below. Both the writing of the information and the reading of the information are usually accomplished according to the present invention using visible light. However, it should be understood that writing of the information may be accomplished by irradiating the active medium with light in the ultraviolet regions, while the reading may be done by light in the infrared region, or may be detected by measuring Raman scattering. Such a reading process at a low energy does not heat the system and does not destroy the stored information.

As mentioned, the information stored by the apparatus of the present invention is stored as a series of data units. According to one embodiment, the data units are binary digits, and each portion of the active medium comprised in the volume represents a 0 or a 1. In this case, there is set a high isomeric ratio threshold and a low isomeric ratio, and volume portions having a isomeric ratio above the high ratio threshold represent 1 digit, while portions having a isomeric ratio below the low ratio threshold represent the other digit. For example, a volume portion having 70% or less active medium of the first isomeric form may represent 0, while a volume portion having 80% or more active medium of the second isomeric form may represent 1. Alternatively, the data representation is analog, and each concentration ratio represents a predefined data unit.

The compounds of formula (II) are stable at room temperature and higher in each of their geometrical state (cis or trans). At higher temperatures interconversion is more rapid, according to the Arrhenius equation. Each of the isomeric structures (of 4,4′-dimethyl-α,α-dicyanostilbene) is stable for a long period (ca. years) in a temperature of up to 35° C. At a temperature of 50° C. interconversion is faster and after about 6 months data is lost. The following Table illustrates stability vs. writeability values for various compounds of formula (II):

| Compound | Lifetime | | writeability |
|---|---|---|---|
| 4,4′-dimethyl-α,α- | >100 | years | write only |
| dicyanostilbene | | | (difficult erasing) |
| 4,4′-dimethoxy-α,α- | Ca. 20 | years | Rewriteable |
| dicyanostilbene | | | (slower erasing) |
| 3,3′4,4′-tetramethoxy-α,α- | Ca. 5 | years | Rewriteable |
| dicyanostilbene | | | (faster erasing) |

The 3-dimensional memory of the present invention may be of a type of “write once” or a rewriteable memory. A precise control of each desirable type of memory may be obtained since the chemical structure of the memory-active compounds dictates its nature. For the case of cis-trans geometric forms, the chemical nature of the substituents on the double bond dictate different stability of each isomeric form and also ease or difficulty in “writing”. Thus by choosing the appropriate active compound, the nature of the memory, whether a “write once” or rewriteable memory may be controlled. It should be understood that heating or irradiating the entire memory can be a process for erasing the stored memory. The binding to the polymer of the active compounds (of formula II or III) results in a well-structured 3-D memory. The polymer further gives physical support and durability to the memory. The chemical and physical properties of the resulting polymer vary and depend on the various active compounds (chromophores), additives and reaction parameters in the polymerization reaction. Temperature gradient, pressure, initiator, duration of polymerization and addition of plasticizer(s) or additional polymers enable a precise control of the desired polymer. In order to eliminate any effects the structurally supporting polymer may exert on the bound compounds and in order to maintain the chemical characteristics of the active bound compound, a chemical spacer is used. Put in other words the present invention provides a three-dimensional memory apparatus for storing information in a volume comprising an active medium made of compounds of formula (II) or (III). Consequently, a memory comprising of compounds of formula (II) or (III) as the active medium is capable of changing from a first isomeric form to a second isomeric form and back as a response to a light irradiation at a first excitation energy, wherein the concentration ratio between the first and the second isomeric forms in a given volume portion represents a data unit; said memory apparatus being characterized in that said active medium comprises compounds according to the invention.

The compounds of formula (II) of the present invention, some of which being effective donor-acceptor-donor, are of the formula:

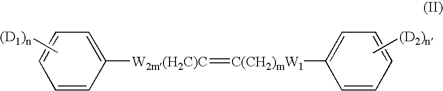

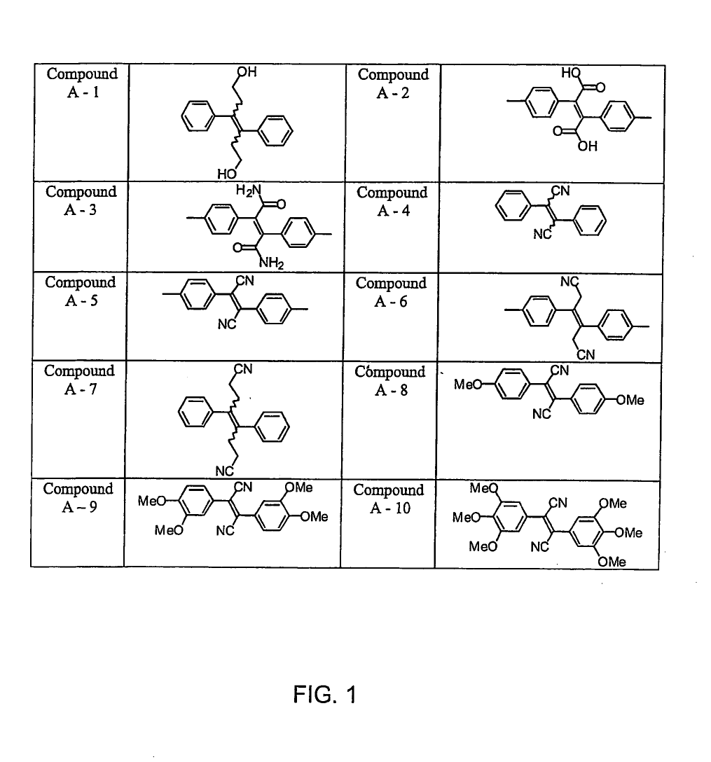

According to a preferred embodiment, the compounds of formula (II) may be those wherein n=n′=0; m=m′=0 or 2 and W₁=W₂ are CN or OH, or may be those wherein n and n′ are 1, 2 or 3; m and m′ are 0 or 1; D₁ and D₂ are R or OR, wherein R is C₁-C₄ allyl; and W₁ and W₂ are CN, COOH or CONH₂. Turning to FIG. 1 there are displayed several examples of compounds of formula (II) that may form the active medium of a 3-dimensional memory as described.

In particular it should be understood that the compounds of formula (II) are actually photoisomerizable donor-acceptor-donor (DAD) molecules, which can be interconverted between isomerization states by two-photon absorption. Stilbene itself (1) is already known to have a high two-photon cross-section but still requires substantial effort to photointerconvert its two isomers. In order to increase its nonlinear absorption characteristics, nitrile groups are attached to its central double bond (making a good acceptor), and various numbers of methoxy groups to the phenyl rings (making good donors). Other compounds of formula (II) according to the present invention may have the general formula: X-α,α-dicyanostilbene, where X is either: 4,4′-dimethyl (2), 4,4′-dimethoxy (3), or 3,3′,4,4′-tetramethoxy (4). These compounds are all transparent to radiation with energy less than 450 nm. The donor-acceptor nature of these molecules is seen visually by the existence of a charge-transfer band in the near-ultraviolet of the absorbance spectrum, which tails off in the visible region leading to a yellow color. This absorbance band is found at longer wavelengths in stronger DAD molecules. For example, 4,4′-dihydroxy-α,α-dicyanostilbene is yellow, while its bispotassium salt (stronger donors) is dark red (longer wavelength absorbance). Analysis of samples at a range of concentrations show that this absorbance obeys Beer's law, thus it is indeed intramolecular and not intermolecular, and also shows that the molecules are not aggregated. Irradiation of the trans-isomers of these compounds with a laser at 460 nm, providing two-photon absorbance at an energy of 230 nm, results in conversion to the cis-isomer to a degree of: 0% (1), 18% (2), 33% (2). Similar irradiation at a lower energy of 514 nm, providing two-photon absorbance at energy of 257 nm gave conversions of 18% (2), 27% (3). Irradiation of the cis-isomers at 600 nm gave no conversion of (1) and only a few percent conversion of (2), but 18% conversion of (3) to the trans-isomer. All these results indicate that a stronger DAD-architectures results in better interconversion of photoisomers (the ‘writing’ and ‘erasing’ processes in a 3D optical memory). The ‘reading’ process, whereby the photoisomeric state of the data is measured also needs to be a multiphoton process, thus the DAD chromophores will also make this more facile. This will result in an intense enough signal to read data at the speeds that are necessary for high-definition video applications.

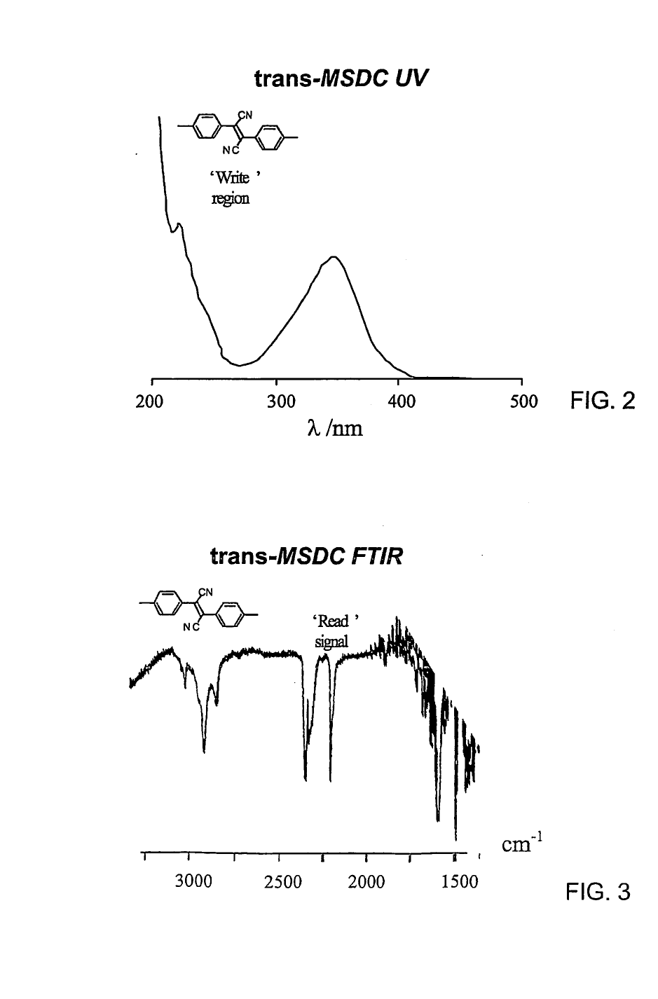

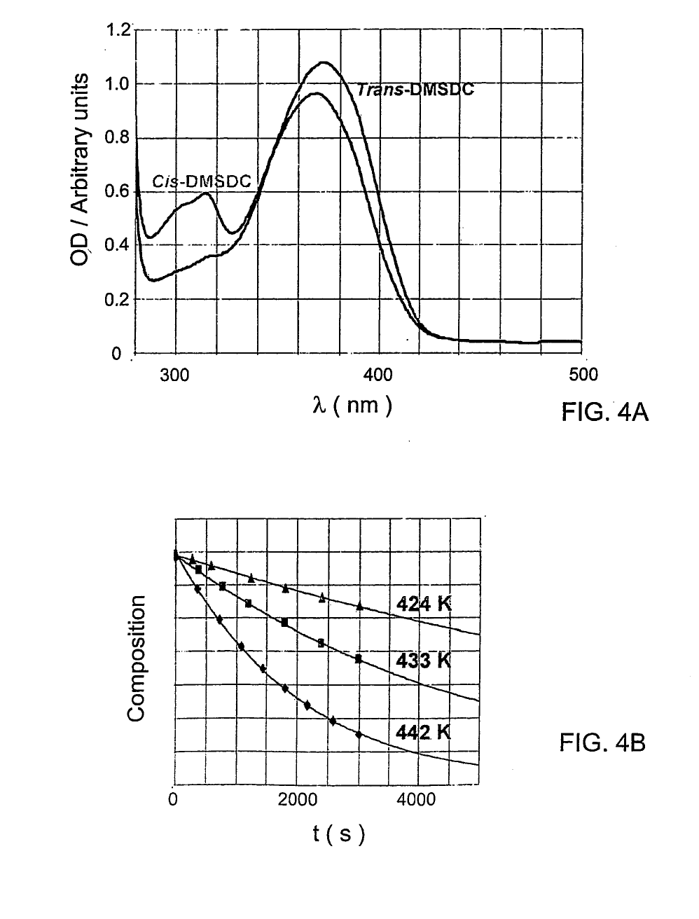

All of the compounds of formula (II), whether in the cis geometry or the trans geometry, upon irradiating the medium comprising these compounds with the appropriate ultraviolet radiation, may interconvert from one geometric structure to the other. Such a transition in the medium is the “writing” process on the memory medium. An example of the possibility of “writing” in the active medium, i.e. exerting a change in the chemical structure of a compound of formula (II) from trans to cis is demonstrated in FIG. 2. In the figure there is shown an ultraviolet-visible spectrum of the compound 4,4′-dimethyl-α,α-dicyanostilbene. The spectrum actually demonstrates the action of “writing” in the memory, since it results in the conversion of a a trans isomer into a cis isomer. Reading the stored information is done at different wavelengths than the writing, where in the reading process the geometrical state of the “written” information is determined with out distorting it. It may be done by InfraRed irradiation or Raman spectrum. FIG. 3 displays a “read” region where the infrared spectrum of the trans-4,4′-dimethyl-α,α-dicyanostilbene is given. As mentioned above, the compounds, which are the active part of the memory, are stable for long periods of time. This stability may be measured by means of spectroscopy. FIG. 4A displays an ultraviolet spectrum of cis- and trans-4,4′-dimethyl-α,α-dicyanostilbene. Thermodynamic equilibrium between states is obtained at a given temperature and measured by NMR in order to elucidate the equilibrium constant and as a result the energy difference between the two states. The equilibrium constant for 4,4′-dimethyl-α,α-dicyanostilbene K₄₄₂^{°}_{K}=0.37, and since ΔG=−RTlnK, ΔG at 442° K is equal to 15.3 kCal/mol⁻¹. Such a value is comparable to literature values of related compounds. The activation energy for the transformation between the two states is calculated by determining the rate of the reaction by NMR at various temperatures, results of which are shown in FIG. 4B.

The preferred compounds of fromula (II) are synthesized by reacting a substituted or non-substituted benzil

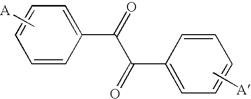

Wherein A and A′ are H, halogen or OR, R being a C₁-C₄ alkyl group; with a BrCH₂C(O)OCH₂CH₃ to yield a compound of formula (W) which may further be reacted to yield a compound of formula (V): 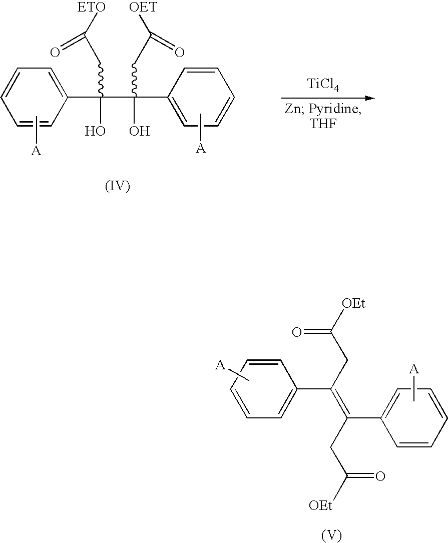
Substituted benzils may be obtained by reacting substituted or unsubstituted benzoyl chloride with substituted or unsubstituted benzene via a Friedel-Crafts reaction to yield appropriately substituted 2-phenyl acetophenone, which may be oxidized to yield a symmetrical or nonsymmetrical benzil.

Compounds of formula (IV) may be reduced to yield a compound of formula (VI), which can further be reacted to yield a compound of formula (VII):

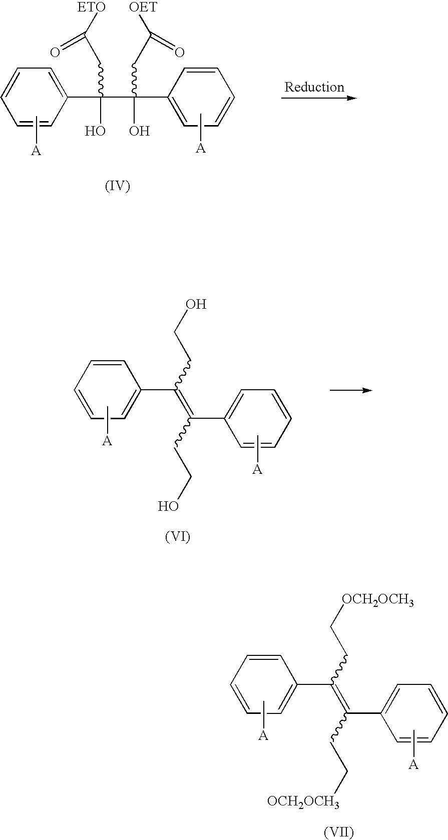

Compounds of formula (II) wherein W₁ and W₂ are CN may be obtained by further reacting compounds of formula (VII) to yield the desired compound of formula (IX).

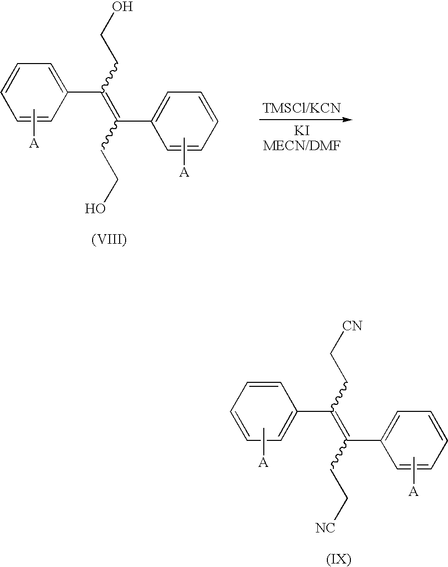

A compound of formula (X) may be obtained by reacting a non-substituted benzoylcyanide in a McMurry reaction:

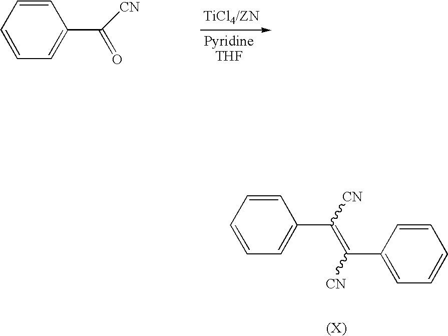

A substituted compound, i.e. of formula (XI) may be obtained by coupling two substituted benzoylcyanide:

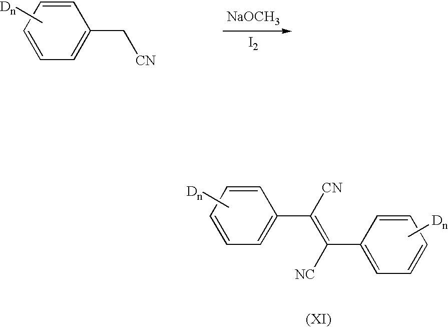

D may be nitro, halogen, R or OR, wherein R is a C₁-C₄ alkyl group and n is 1, 2 or 3. In the case R is a CH₃ group, a benzylic hydrogen may be substituted by a halogen using the appropriate N-halogenyl succinamide to yield a compound of formula (XII).

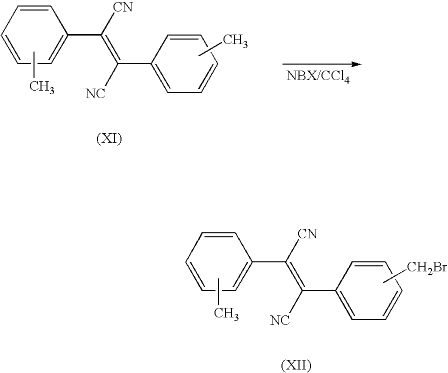

The 3-dimensional optical memory of the present invention is composed of compounds of formula (I): 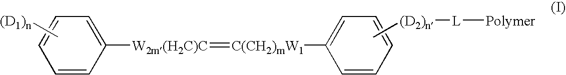
wherein m and m′ are independently 0, 1 or 2. n and n′ are independently 0, 1, 2, or 3. W₁ and W₂ are independently selected from CN, OH, C═CR, COOH, COOR wherein R is a C₁₋₄-alkyl group, CONH₂, OCH₂OCH₃. D₁ and D₂ are independently selected from R, NO₂, halogen or O—R wherein R is a hydrogen, C₁₋₄-alkyl group optionally substituted by halogen. L is a linking group selected from (CH₂)ₐX or O(CH₂)_{b}X, a and b being 0-10 and X being O—C(═O)C—. It should be understood that L may be the core of any bi-functional bridging group whose functional groups are capable of attaching by chemical means to the compound of formula (II) and to the polymer, e.g. OH, O—C(═O)C═CH₂, halogen. The polymer may be selected from the group of poly(alkyl metacrylate)s and their copolymers, or polystyrene and its copolymers. More specifically the polymer is poly(methyl metacrylate).

The polymer may be a homopolymer where to the basic skeleton of the polymer are attached as side-chains the active compounds (chromophores) of fromula (II) used for interactions with the incident light. Another option is to produce a copolymer. In such a case a compound of formula (II) is first converted by chemical means into a polymerizable compound, i.e. a monomer, without effecting its activity with light. The resulting light-active monomer is then polymerized in the presence of another monomer to form a copolymer having active compounds as part of its skeleton.

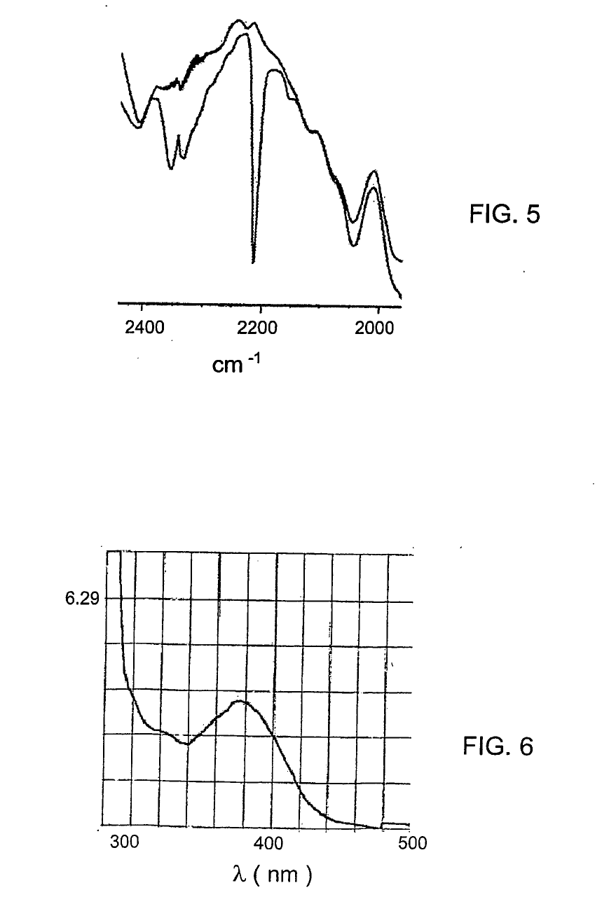

Turning to FIG. 5 there is shown the infrared spectrum of a compound of formula (II) bound to a polymer, i.e. (II)—L-P. The compound of formula (II) is 4,4′-dimethoxy α,α-dicyanostilbene, and the polymer is polymethylmethacrylate (PMMA). The binding is done through a spacer (L) and thus the bound active compound comprises only of one free methoxy group and an OR group. The spectrum comprises of only a single absorption for the CN group, clearly demonstrating that the cyano groups in the α-positions are unaltered in the course of the chemical binding of the compound of formula (II) to the bi-functional spacer and subsequently to the polymer. FIG. 6 shows an ultraviolet spectrum of the polymer-bound 4,4′-dimethyl-α,α-dicyanostilbene through a spacer. The concentration of the chromophore may be calculated (ca. 0.1%).

The preferred polymers comprising the compounds of formula (II) are synthesized by derivitizing a compound of formula (II) and subsequently reacting the derivitized compound with a bi-functional spacer and the resulting compound is reacted with a polymer. Thus reacting a compound of formula (XII) with a bifunctional spacer to yield a compound of formula (XIII):

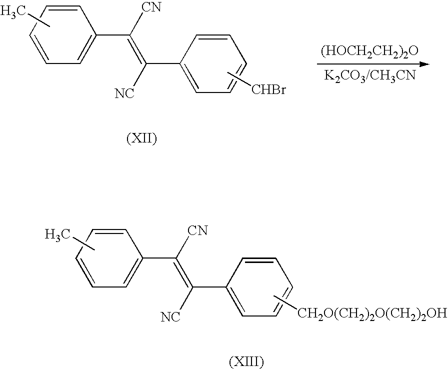

A transesterification reaction of a compound of formula (XII) with a polymer yields a compound of formula (XIV):

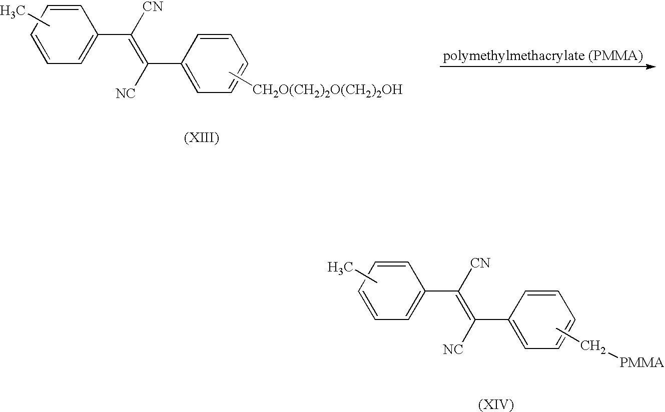

Alternatively, the compound of formula (I) may be obtained by reacting a compound of formula (II) to from a derivitized compound. The derivitized compound is then reacted with a bi-functional spacer to form an appropriate monomer, which is polymerized in the presence of a monomer to yield a copolymer. Thus a compound of formula (II) is reacted to yield a compound of formula (XV): 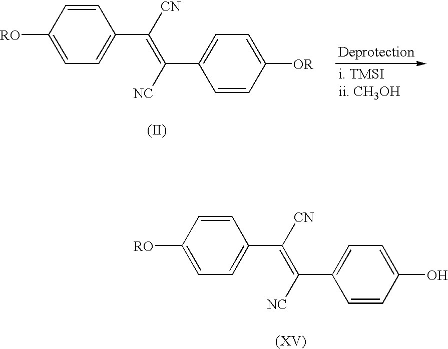
R is a C₁₋₄-alkyl. The appropriate spacer is prepared according to the following scheme to yield a bi-functional compound of formula (XVI): 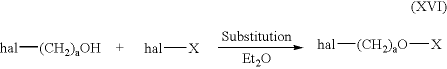
   where a and X are as defined above. In the next step the bi-functional spacer (XVI) is reacted with the compound of formula (II) to yield a compound of formula (XVII):

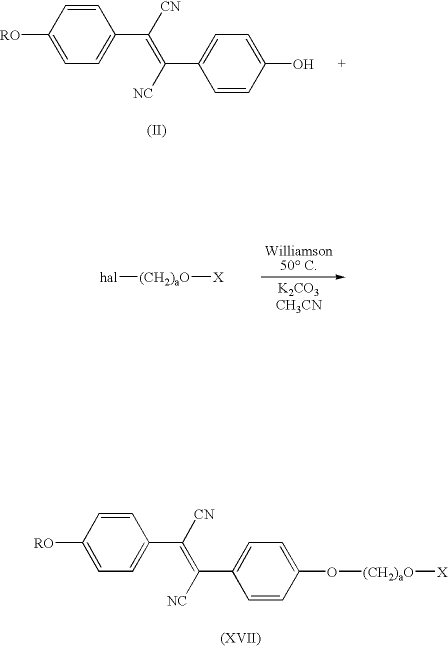

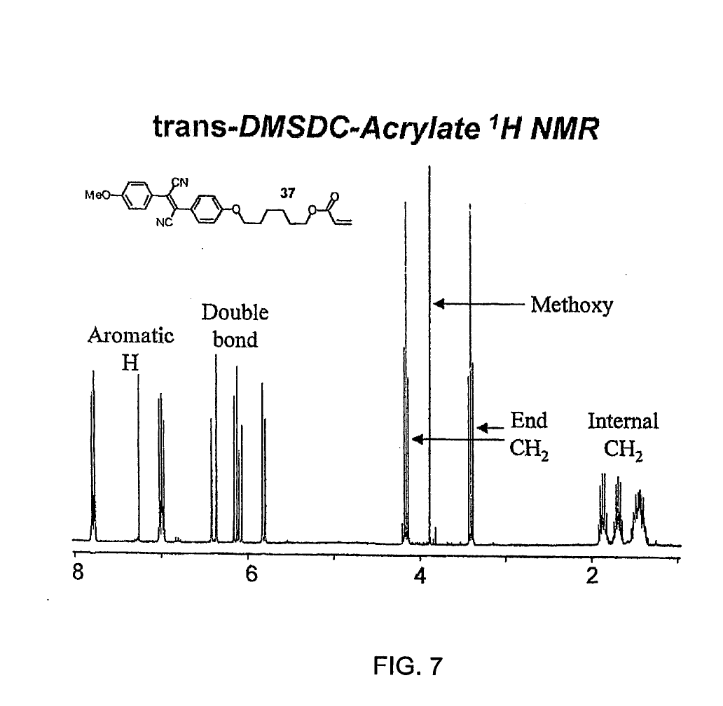

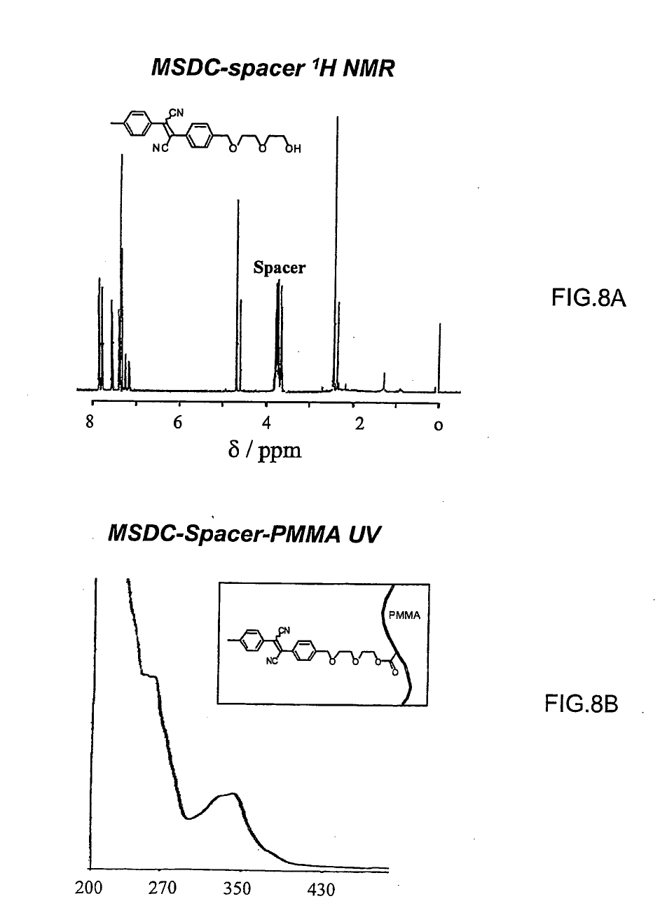

The NMR spectrum of the compound (XVII), wherein R is an alkyl group, a is 6 and X is C(═O)CH═CH₂ is given in FIG. 7. The compound of formula (XVII) is subsequently polymerized in the presence of MMA to yield a copolymer of formula (XVIII):

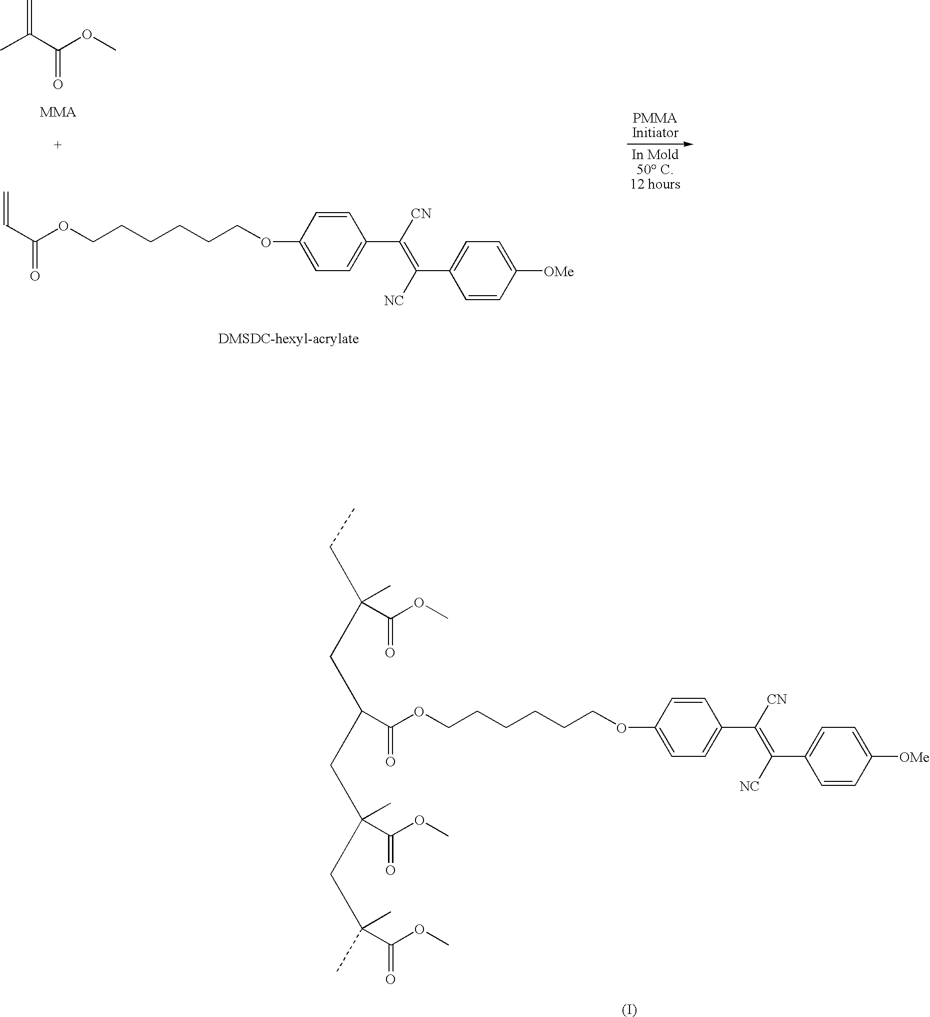

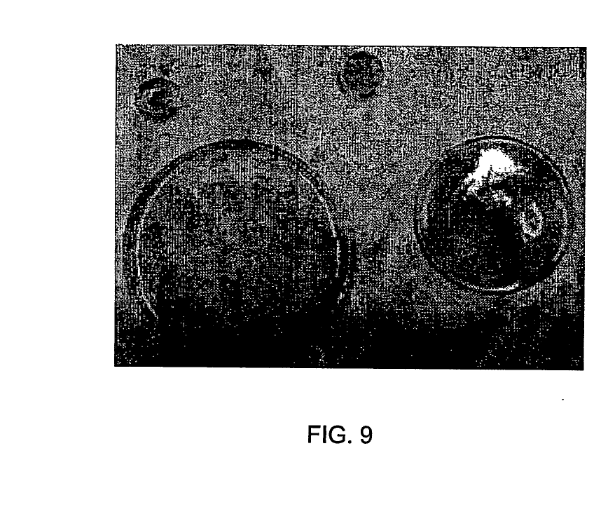

FIG. 8 shows a ultraviolet spectrum of a compound of formula (XVIII) wherein 4,4′-dimethyl-α,α-dicyanostilbene bound through diethylene glycol as a spacer to PMMA in its two isomeric states cis and trans, i.e. used in the memory of the present invention as ‘o’ and ‘1’ binary states. Turning to FIG. 9 there is presented a picture of a 3-dimensional memory unit of the present invention in the form of a disc. The disc is composed of the active compound 4-methoxystilbene-α,α-dicyanide bound to a PMMA through a spacer.

### EXAMPLES

### Example 1

### 4-Bromobenzil

AlCl₃ (13.3 g, 0.1 mol) was added to stirring, degassed bromobenzene (150 mL) at 0° C., under argon. Benzoylchloride (15.4 g, 0.1 mol, as obtained from Aldrich) was slowly added by syringe, then the reaction was allowed to stir for 12 h while it warmed to ambient temperature. The reaction was finally heated to 100° C. for 1 h, and was then quenched by pouring onto a mixture of ice (200 g) and conc. HCl (20 mL). The organic layer was combined with one extraction of the aqueous layer (toluene, 100 mL), and was then washed with 3M NaOH (100 mL) and water (100 mL×2). The crude product was isolated by drying of the solution over MgSO₄, filtration, and evaporation of the solvent. An orange solid was obtained (2-phenyl-p-bromoacetophenone), which showed one major product (Rf=0.53 in 1:1 DCM:hexane) and a slower trace impurity. It was used without further purification.

Crude 2-phenyl-p-bromoacetophenone (assume 0.1 mol) was suspended in 70% AcOH (250 mL) at ambient temperature, and SeO₂ (12.1 g, 0.11 mol) was added. The mixture was brought to reflux, upon which the starting material dissolved, and several colour changes were observed over 12 h, culminating with a clear yellow solution with a black precipitate. The finished reaction was poured onto water (250 mL), and the mixture was cooled in ice. The precipitate was collected, dissolved in ether, dried over Ca₂CO₃, and filtered, and then the solvent was evaporated to give the crude product (4-bromobenzil). A yellow solid was obtained (Rf=0.58 in 1:1 DCM:hexane), which showed several slower-moving trace impurities. Yield over 2 steps: 25.71 g=89%. It was used for later steps without further purification.

### Example 2

### Reformatsky Reaction of 4-bromobenzil

Dimethoxymethane (50 mL, freshly distilled) was poured on zinc granules (150 g, 150 mmol), and then ethylbromoacetate (16.63 mL, 150 mmol) was added by syringe, slowly enough to keep the reaction under control. The mixture was stirred under reflux for 1 hour, after which almost all the zinc had been consumed, then was allowed to cool to below reflux temperature. 4-Bromobenzil (8.67 g, 30 mmol) in DMM (50 mL) was then added dropwise via a pressure-equalized dropping funnel over 30 mins, and the reaction was refluxed for 2 h. After cooling to ambient temperature, the reaction was quenched with water (50 mL), then was introduced into a separating funnel along with ether (50 mL) and 25% H₂SO₄ (50 mL). The organic layer was combined with one ether extraction (50 mL) of the aqueous layer, was dried over MgSO₄, filtered, and the solvents were evaporated along with excess and hydrolyzed ethylbromoacetate to yield the meso compound of formula (H). A slightly yellow solid was obtained (Rf=0.71 in EtOAc), with a very slightly faster by-product (possibly the R,R and S,S isomers) and some slower trace impurities. Crude yield: 16.7 g, the crude product was used without further purification.

### Example 3

### 4-bromostilbenediethylacetate

TiCl₄ (5.04 mL, 40 mmol) was added dropwise by syringe to stirring, freshly distilled THF (100 mL), giving a bright yellow suspension. Zinc dust (5.23 g, 80 mmol) was then added portionwise, noting the appearance of the black Ti salts. The mixture was stirred under reflux for 2 h, then was allowed to cool. Pyridine (2.5 mL) was added by syringe, then the material obtained in Example 2 (5.56 g, theoretically 10 mmol) in THF (25 mL) was added via a pressure-equalized dropping funnel. The reaction was stirred at ambient temperature under N₂ for 3 days, after which it had a deep red-brown color. Finally, the reaction was stirred under reflux for 2 h, before being cooled and slowly quenched with 20% conc. HCl (100 mL) added via a pressure-equalized dropping funnel. The purple mixture was extracted with ether (2×50 mL), and the extractions were dried over copious Na₂CO₃ then condensed to give a crude yellow solid (4.8 g). This product was subjected to column chromatography (DCM on silica gel) to give pure 4-bromostilbenediethylacetate. A light yellow oil containing only one isomer was obtained (Rf=0.41 in DCM). Yield 1.16 g=27% from 4-bromobenzil.

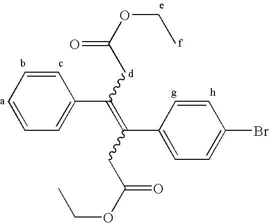

¹H NMR (CDCl₃)

| | |
|---|---|
| abcgh | m, 6.90, 7.00, 7.09, 7.20 |
| d | s, 3.59, 3.60 |
| e | m, 4.08 |
| f | m, 1.17 |

### Example 4

### 4-Bromostilbenediethyloxymethoxymethane

Solid LiAlH₄ (142 mg, 4 mol eq.) was slowly added to a stirring solution of the compound obtained in Example 3 (580 mg, 1.35 mmol) in diethyl ether (15 mL) in an ice bath. After the reaction had subsided, the ice bath was removed and the reaction was stirred for a further 2 h, after which it was quenched by the slow addition of 1M HCl (10 mL). The ether layer was taken along with an EtOAc extraction of the aqueous layer (15 mL), was dried over Mg₂SO₄, filtered, and concentrated to give crude 4-bromostilbene diethanol (399 mg, theoretical 85%). The crude 4-bromostilbene diethanol (theoretical 1.35 mmol) was dissolved in dry dimethoxymethane (25 mL), and LiBr (59 mg, 0.5 mol eq.) and tosic acid (58 mg, 0.25 mol eq.) were added. After stirring for 1 h at ambient temperature, further LiBr (12 mg, 0.1 mol eq.) was added, then stirring was continued for a further 18 h. Water (25 mL) and ether (25 mL) were added, and the organic layer was taken along with one extraction (ether, 25 mL) of the aqueous layer. The combined organic solutions were dried over Mg₂SO₄, filtered, and condensed to give a crude product (415 mg), which was purified by column chromatography (DCM with 0-10% EtOAc on silica gel) to give pure 4-Bromostilbenediethyloxymethoxymethane. A colorless oil was obtained (Rf=0.72 in 17:3 DCM:EtOAc). Yield: 271 mg=46% over 2 steps.

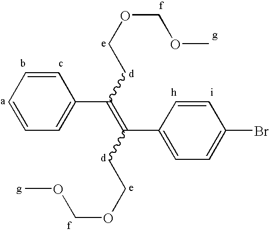

¹H NMR (CDCl₃)

| | |
|---|---|
| abc | m, 6.92 (2H), 7.08 (3H) |
| d | t, 2.89, 2.91 |
| e | t, 3.49, 3.49 |
| f | s, 4.56, 4.56 |
| g | s, 3.32, 3.33 |
| hi | m, 6.81, 7.18 |

### Example 6

### Stilbene Diethanol

Benzil (6.27 g, 30 mmol) was reacted using a Reformatsky reaction as for 4-bromobenzil (Example 1), using, Zn (9.8 g, 150 mmol) DMM (150 mL) and ethylbromoacetate (11 mL, 110 mmol). Next, a McMurry reaction was carried out as described previously, but using TiCl₄ (15 mL, 120 mol. eq;), Zn (15.7 g, 240 mmol), pyridine (7.5 mL) and THF (150 and 100 mL). The crude product was not purified further. The reduction was carried out as described previously, using LiAlH₄ (3.0 g) and ether (150 mL). The crude product (10.3 g) was purified by column chromatography (1:1 EtOAc:hexane, then pure EtOAc on silica gel) to give pure stilbene diethanol. A white crystalline solid containing only one isomer was obtained (Rf=0.41 in EtOAc). Yield: 3.63 g=45% over 3 steps.

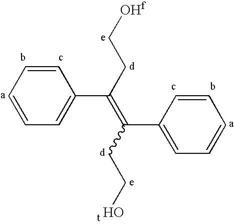

¹H NMR (CDCl₃)

| | |
|---|---|
| abc | m, 6.9-7.2 |
| d | t, 2.88 |
| e | t, 3.67 |
| f | s, x.xx |

### Example 7

### Stilbene Dipropionitrile

Stilbene diethanol (640 mg, 2.4 mmol), ground KCN (480 mg, 12.5 mmol), and KI (ca. 10 mg) were suspended in a mixture of MeCN (5 mL) and DMF (5 mL). The mixture was degassed and left under a slow flow of nitrogen, which was bubbled through NaOH to neutralize evolved HCN. TMSCl (0.76 mL, 12.5 mmol) was then added by syringe through a septum, and the reaction was heated to 60° C. for 5 hours. After cooling, the mixture was poured on 0.1 M NaOH (50 mL), which was extracted with chloroform (50 mL×3). The combined ectractions were combined, dried, filtered and condensed to give a crude product that was purified by column chromatography (9:1 DCM:EtOAc on silica gel). A white crystalline solid containing only one isomer was obtained (Rf=ca. 0.4 in 9:1 DCM:EtOAc). Yield: 15 mg=2%.

### Example 8

### Stilbene Dicyanide

TiCl₄ (25 mL, 0.2 mol) was added dropwise by syringe to stirring, freshly distilled THF (250 mL), giving a bright yellow suspension. Zinc dust (13.8 g, 0.2 mol) was then added portionwise, noting the appearance of the black Ti salts. The mixture was stirred under reflux for 2 h, then was allowed to cool. Pyridine (10 mL) was added by syringe, then benzoylcyanide (13.1 g, 0.1 mmol) in THF (50 mL) was added via a pressure-equalized dropping funnel. The reaction was stirred at reflux under N₂ for 2 h, bringing a deep blue color, before being cooled and slowly quenched with 10% conc. H₂SO₄ (10%, 150 mL) added via a pressure-equalized dropping funnel. Water (200 mL) was added, then the mixture was extracted with ether (3×200 mL), the extractions were dried over copious Na₂CO₃, then were condensed to give a crude yellow oil. This product was subjected to column chromatography (1:1 hexane:DCM, then DCM on silica gel) to is give pure stilbene dicyanide. A light yellow oil was obtained (Rf=ca. 0.5 in DCM). Yield was not determined.

### Example 9

### Methylstilbene Dicyanide (MSDC)

4-Methyl benzylcyanide (13.2 mL, 0.1 mol) and I₂ (25.4 g, 0.1 mol) were dissolved in dry ether (300 mL) at 0° C. A freshly prepared solution of sodium (4.7 g, 0.2 mol) in MeOH (50 mL) was then added over 30 minuts, during which time the solution lost its color and a precipitate formed. The product was collected and washed with ether. Additional material was obtained by condensing the supernatant. A colourless solid was obtained (Rf=ca. 0.8 in DCM). Yield 12.8 g (99%).

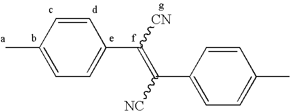

| | |
|---|---|
| ¹H NMR (CDCl₃) | ¹³C NMR (CDCl₃) |

| | | | |
|---|---|---|---|
| a | s, 2.46 | a | 21.5 |
| cd | m, 7.35, 7.73 | be | 124.5, 129.3 |
| | | cd | 128.5, 129.8 |
| | | f | 142.2 |
| | | g | 116.9 |

### Example 10

### 4-bromomethyl Stilbene Dicyanide

N-Bromo succinimide (2.4 g, 1.1 mol eq. mmol) and methylstilbenedicyanide (3.2 g, 12.4 mmol) were dissolved in refluxing CCl₄ (50 mL). A catalytic quantity of benzoyl peroxide was added, and the reaction was stirred under reflux for 2 h. No exothermism was noted. After cooling, the reaction mixture was condensed and the crude product was purified by column chromatography. A large quantity of methylstilbenedicyanide remained unreacted. A colorless solid was obtained (Rf=ca. 0.7 in DCM). Yield ca. 200 mg=ca. 5%.

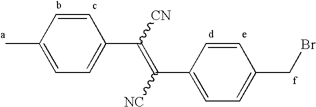

¹H NMR (CDCl₃)

| | |
|---|---|
| a | m, 2.37, 2.38 |
| bcde | m, 7.19, 7.23, 7.32, 7.35 |
| f | m, 4.46 |

### Example 11

### 4,4′-dimethoxy-α,α-dicyanostilbene

Sodium metal (17 g) was dissolved in MEOH (150 mL), and the resulting solution was added over 2 hours to a stirring solution of (4-methoxyphenyl) acetonitrile (50 mL, 0.37 mmol), THF (250 mL) and I₂ (93 g) at −5 C, under an inert atmosphere. The yellow mixture was then stirred a further 15 minutes, after 1 which the solvents were removed under vacuum. The resulting solid was partitioned between DCM (500 mL) and 0.025 M sodium thiosulfate (400 mL). The organic layer was collected, combined with 2 extractions (100 mL) of the aqueous layer, dried over magnesium sulfate, filtered, then finally condensed to ca. 50-100 mL. The yellow crystals were filtered off and washed with ether, giving pure trans (20.5 g, 38%). The remaining solution was condensed, then MeOH (100 mL) was added. More crystals formed, which were collected giving cis (10.8 g, 20%). The remaining solution was condensed and chromatographed to give additional cis (14.5 g, 27%).

Total Yield=86%. Analyses for trans-isomer: ¹H NMR: m, 7.79; m, 7.01; s, 3.88. 13C NMR: 162.0; 130.4; 124.6; 122.7; 117.3; 114.6; 55.5. EA: Expctd (C 74.47, H 4.86, N 9.65), Rcvd (C, 74.27; H, 4.83; N, 9.61).

### Example 12

### 3,3′,4,4′,5,5′-hexamethoxy-α,α-dicyanostilbene

3,4,5-trimethoxybenzylnitrile (25 g) was mixed with iodine (43 g, 1 mol. eq.) in ether (500 mL) and the solution was cooled to 0° C. A solution of sodium (7.9 g, 1 mol. eq.) in MeOH (100 mL) was added dropwise, after which much of the color had been lost, and a precipitate had formed. The precipitate was collected and washed with ether and water. The supernatant and washings were combined, condensed, and water (300 mL) and DCM (300 mL) were added. The DCM layer was taken along with one extraction (100 mL), dried, filtered, and the solvent was removed. The precipitate gave pure trans-3,3′,4,4′,5,5′-hexamethoxy-α,α-dicyanostilbene.

### Example 13

### 4,4′-dihydroxy-α,α-dicyanostilbene

4,4′-dimethoxy-α,α-dicyanostilbene (example 11) (20.0 g, 35 mmol) and NaI (20 g) were suspended in toluene (500 mL) under an inert atmosphere, then pyridine (20 mL) and AlCl₃ (20 g) were added. The reaction was protected from is light and stirred at reflux for 2 days. The finished reaction was decomposed with 10% HCl (200 mL) while hot, was cooled, then the crude product was collected by filtration and recrystallized from MeCN. Pure compound is obtained (17.2 g, 96%). ¹H NMR: s, 9.21; m, 7.74; m, 7.02. 13C NMR: 161.0; 131.4; 124.9; 123.6; 118.1; 116.8. EA: Expctd (C, 73.27; H, 3.84; N, 10.68), Rcvd (C, 73.36; H, 3.99; N, 10.94).

### Example 14

### Hydrolysis of 4,4′-dimethyl-α,α-dicyanostilbene

4,4′-dimethyl-α,α-dicyanostilbene (300 mg) was hydrolized by reflux with KOH (280 mg) in EtOH (30 mL). A 3 h reflux yields the diamide (A), while a 18 h reflux yields the carboxylic acid (B).

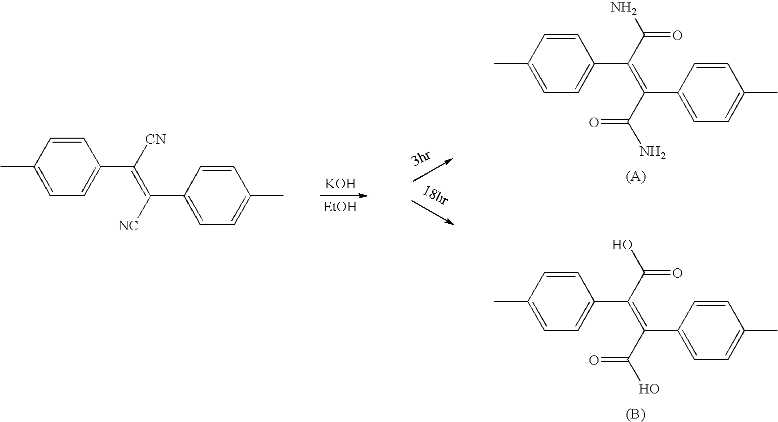

¹HNMR:
(A): δ 2.34, 7.1, 7.12, 7.33, 7.35.
(B): δ 2.37, 7.15, 7.18, 7.36, 7.39.

### Example 15

N-Bromo succinimide (2.9 g, 1.3 mol eq. mmol) and 4,4′-dimethyl-α,α-dicyanidestilbene (3.2 g, 12.4 mmol) were dissolved in refluxing CCl₄ (25 mL). A catalytic quantity of benzoyl peroxide was added, and the reaction was stirred under reflux for 3 h, during which time flier benzoyl peroxide was added every 30 minutes. After cooling, DCM (25 mL) was added, the mixture was filtered, and the filtrate was washed well with DCM. The solvents were removed under vacuum, and the product was isolated by column chromatography on silica gel (1:1 hexane:DCM, then DCM). Colorless solids were obtained (Rf=ca. 0.2 and 0.3 in 1:1 hexane:DCM). Yield 1.55 g (trans) and 1.09 g (cis)=ca. 63%.

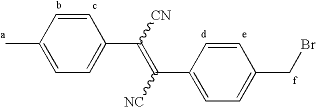

¹H NMR (CDCl₃)

| | trans | cis |
|---|---|---|
| a | m, 2.45 | m, 2.37 |
| bcde | m, 7.35, 7.56, 7.75, 7.81 | m, 7.19, 7.23, 7.32, 7.35 |
| f | m, 4.46 | m, 4.46 |

### Example 16

4-bromomethyl-4′-methylstilbene-α,α-dicyano of example 1 (652 mg, 2 mmol) was dissolved in anhydrous MECN (20 mL), and diethylene glycol (2 mL, ca. 10 mol. Eq.) and K₂CO₃ (1.7 g) were added. The reaction was stirred under argon at ambient temperature for 18 h, after which the product had disappeared, as determined by TLC. Most of the solvent was removed under vacuum, then the mixture was dissolved in ether (15 mL) and washed with brine (10 mL×3). The aqueous extractions were extracted with ether (15 mL) to recover more material. The ethereal fractions were combined, dried, evaporated, and subjected to column chromatography (DCM, then EtOAc on silica gel) to isolate the major product (pair of spots, Rf ca. 0.7). A mixture of cis and trans stilbene products was obtained. Yield: 410 mg=59%.

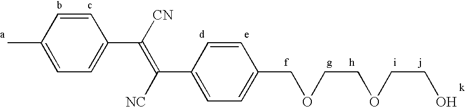

¹H NMR (CDCl₃)

| | |
|---|---|
| a | s, 2.44 |
| bcde | m, 7.33, 7.52, 7.74, 7.81 |
| f | m, 4.66 |
| ghi | m, 3.65-3.8 |
| j | m, 3.6-3.65 |
| k | not observed |

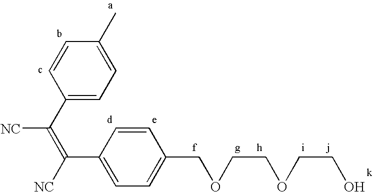

| | |
|---|---|
| a | s, 2.36 |
| bcde | m, 7.12, 7.20, 7.31 |
| f | m, 4.57 |
| ghi | m, 3.65-3.8 |
| j | m, 3.6-3.65 |
| k | not observed |

### Example 17

The trans isomer obtained in Example 3 (50 mg) was dissolved together with 0.15 mL of H₂SO₄ and polymethylmetacrylate (500 mg) in CHCl₃ (3 ml). The reaction mixture was stirred at 60° C. for 18 h. The polymer was precipitated by slowly dripping the solution into swirling CH₃OH (30 mL). The polymer was collected by filtration, redissolved in CHCl₃, filtered, precipitated and dried. Ultraviolet analysis revealed the presence of ca. 0.2% chromophore in the product.

### Example 18

PMMA (1.0 g) was dissolved in chloroform (7 mL) and a solution of Na (80 mg) in diethylene glycol (ca. 3 mL) was added. The reaction was monitored by removing aliquots to monitor the progress of the reaction. Functionalized PMMA at the amount of ca. 5% was obtained after 5 days. The functionalized PMMA obtained was dissolved in dry MeCN (5 mL) and 150 mg of 4-bromomethyl-4′-methyl-α,α-dicyanidestilbene and potassium carbonate (100 mg) were added. This reaction was stirred for 5 days, after which the mixture was filtered and precipitated twice by dripping the reaction mixture into 50 mL CH₃OH, isolation, washing the precipitate with aqueous CH₃OH and drying.

### Example 19

PMMA-co-5%-methacrylic acid (200 mg, 0.1 mmol acid) and the compound obtained in Example 3 (50 mg, ca. 1.5 mol. eq.) were dissolved in CHCl₃ (5 mL) at 0° C. under nitrogen. DCC (36 mg, ca. 1.5 mol. eq.) was added and the reaction was stirred for 24 h during which it warmed to ambient temperature. The solvent was removed under vacuum, then the solid obtained was dissolved in a minimum of acetone. Precipitation of ther product was initiated by the slow addition of CH₃OH, followed by concentration under vacuum. The precipitate was washed well with CH₃OH and dried. A white solid was obtained. UV analysis revealed a chromophore content (by mass) of approximately 1%.

### Example 20

PMMA-co-5%-methacrylic acid (200 mg, 0.1 mmol acid) and diethylene glycol (ca. 0.5 mL) were dissolved in CHCl₃ (1 mL), then DCC (ca. 100 mg) was added. The reaction was stirred for 18 h, then the solvent was evaporated. The crude mixture was dissolved in a minimum of acetone, an equal volume of CH₃OH was added, and the solvents were evaporated. The resulting powder was washed well with CH₃OH then dried under vacuum. The powder (80 mg) was dissolved in dry CH₃CN (3 mL), and 4-bromomethyl-4′-methylstilbene-α,α-dicyanide (25 mg, ca. 1.5 mol. eq.) was added. K₂CO₃ (150 mg) was added, and the reaction was stirred at ambient temperature for 3 weeks. The supernatant and a chloroform washing of the solid were dripped into stirring CH₃OH (50 mL) to precipitate the product, which was further purified by dissolving in chloroform and a further precipitation. The resulting colorless solid was shown by UV spectroscopy to contain ca. 3% of the stilbene component by weight, corresponding to a ca. 20% yield of linked chromophore from acid functionality.

### Example 21

### 4-Hydroxy-4′-methoxy-α,α-dicyanostilbene

4,4′-dimethoxy-α,α-dicyanostilbene (2 g, 6.8 mmol) was dissolved in chloroform (20 mL) under anhydrous conditions, under nitrogen. TMSI (1.67 mL, 1.5 mol. Eq.) was added by syringe, and the reaction was stirred for three days at 50° C. During the reaction, it slowly becomes a dark purple color. Most of the is starting material was recovered. The product is an orange spot, R_{f} 0.1 in DCM, which moves fast in ethyl acetate. Yield, ca. 50 mg=ca. 2%.

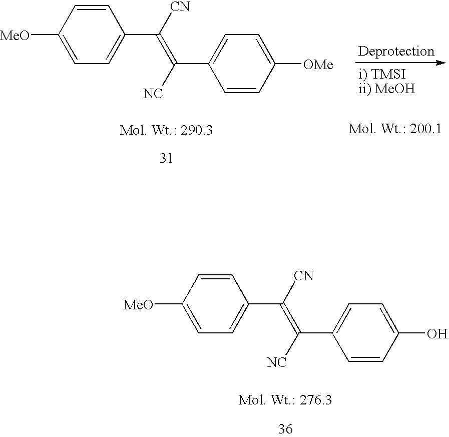

### Example 22

### 4-Hydroxy-4′-methoxy-α,α-dicyanostilbene

4,4′-dihydroxy-α,α-dicyanostilbene (Example 13) (30.0 g) and KOH (7.0 g) were dissolved in acetone (150 mL) under an inert atmosphere. The mixture was brought to reflux, iodoethane (15 mL) was added, and reflux was continued for 3 h, by which time the red reaction mixture had turned orange. The mixture was cooled, sufficient HCl was added to obtain a yellow color, and most of the solvent was removed. The mixture was then taken up in DCM (150 mL), filtered, and the solid was washed with DCM. [The solid was washed with water and dried to give recovered starting material]. The DCM solution was evaporated to dryness, then was taken up in 0.5 M NaOH (200 mL). The resulting suspension was filtered and the solid was washed well with water. [The solid is the bis-ethylated product]. Conc. HCl was added to the basic solution until a yellow color was obtained, then the precipitate was collected by filtration, washed with water, and dried to give 46 as a yellow solid (25%). ¹H-NMR (CDCl₃, 298 K, 300 MHz, trans-isomer): m, 7.7-7.8; m, 6.9-7.0; q, 4.1; t, 1.45. ¹³C-NMR (CDCl₃, 298 K, trans-isomer): 162.3; 161.2; 131.5; 131.3; 116.9; 115.8; 64.6; 14.9. EA: Expctd for 46-0.5H₂O(C 72.23; H, 5.05; N, 9.36), Rcvd (C, 72.53; H, 4.99; N, 9.29).

### Example 23

### Bromohexyl Methacrylate

6-Bromo-hexan-1-ol (5 g, 28 mmol) was dissolved in diethyl ether (20 mL), and cooled in ice under nitrogen. Acryloyl chloride (3 mL, 37 mmol) was added, the the reaction was stirred at ambient temperature for 1 hour. The volatile compounds were removed under vacuum, leaving slightly impure compound (designated 35) (5.9 g, ca. 65%).

NMR: m 6.3-6.5, m, 6.1-6.2, m 5.8, t 4.15, t 3.65, m 3.6, m 1.8-1.9

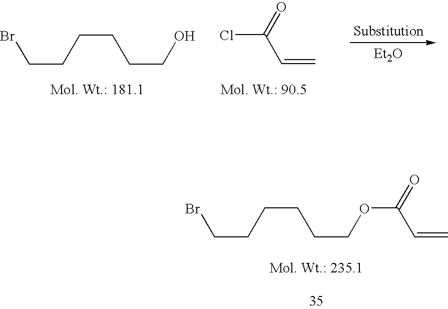

### Example 24

### Bromopropyl Methacrylate

Methacrylic acid (10 mL, 118 mmol) and KOH (6.62 g, 118 mmol) were added to DMF (100 mL), and stirred with heating to 70 C until the KOH was completely dissolved. 1,3-Dibromopropane (25 mL, 2 ca. 2 mol. eq.) was added, and stirring was continued at the same temperature for 18 hours. Most of the DMF and the excess dibromopropane were then removed under vacuum. Hexane was added (50 mL), the inorganic material and polymer was removed by filtration, then the material was again evaporated under vacuum to give the product as a colourless liquid (9.4 g, 38%). ¹H-NMR (CDCl₃, 298 K, 300 MHz): m, 6.10; m, 5.57; t, 4.27; t, 2.50; m, 2.23; m, 1.94.

### Example 25

4-Hydroxy-4′-methoxy-α,α-dicyanostilbene (Example 20 or 21) (ca. 25 mg) and bromohexyl methacrylate (200 mg, ca. 2 mol. Eq.) were dissolved in MeCN (15 mL) under nitrogen. K₂CO₃ (60 mg). The yellow solution slowly turned red at the formation of the phenolate anion. The reaction was heated to 50° C. for 18 hours, after which the color had returned to yellow, indicating the end of the reaction. The to solvent was removed under vacuum, then the mixture was chromatographed (chloroform on silica gel) to give pure methyl-stilbenedicyano-hexyl-methacylate (designated 37) (R_{f}=0.48). Yield ca. 10 mg=ca. 35%.

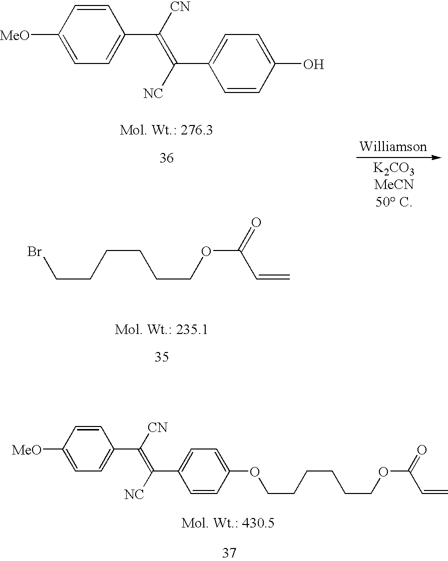

### Example 26

### Copolymerization

methyl-stilbenedicyano-hexyl-methacylate (Example 25) (ca. 3 mg) was dissolved in a few drops of methyl methacrylate. Prepolymerized MMA (3 mL, prepared by heating a filtered 1% solution of benzoyl peroxide in MMA at 60° C. for 2 h) was added and the mixture was shaken lightly to mix. The mixture was heated in a glass tube at 60° C. for 18 hours, after which it had become a hard solid. The glass tube was then broken to release the polymer monolith.

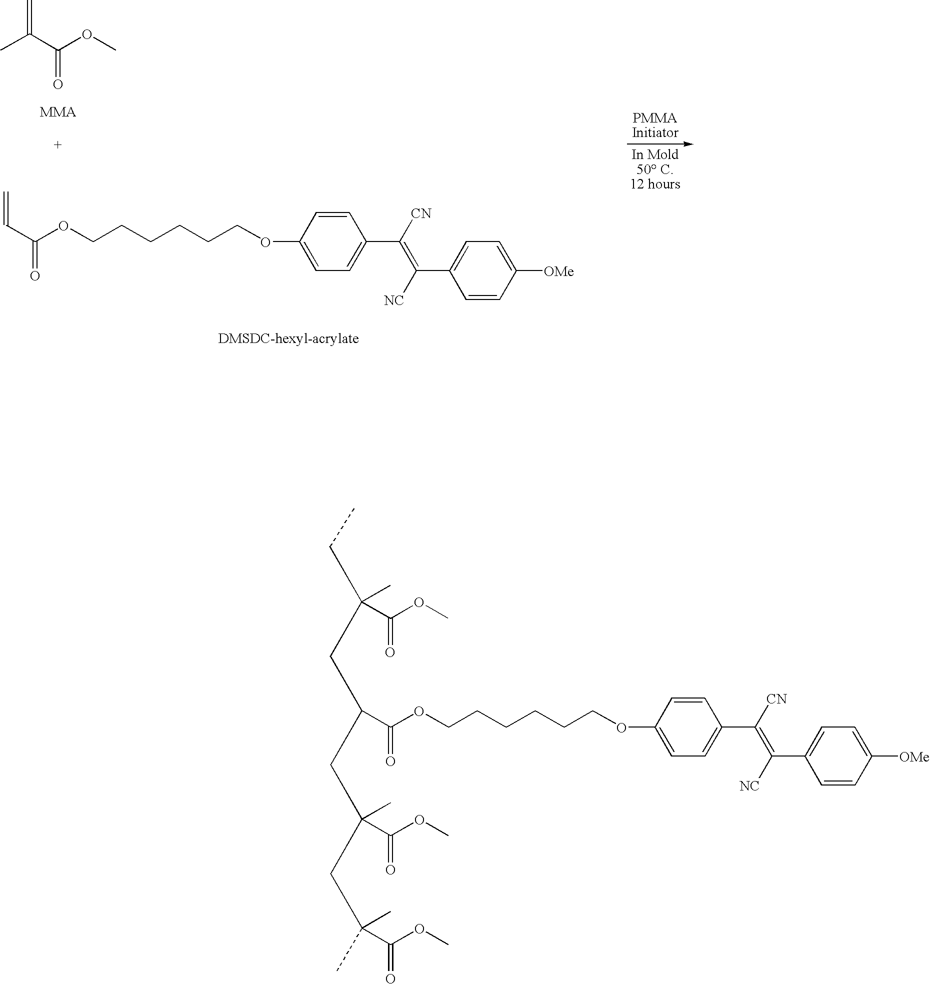

1-25. (canceled)

26. A compound of formula (I) 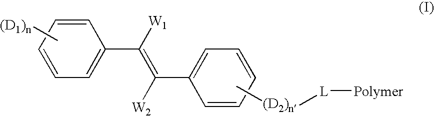
wherein the orientation of the substituents is either cis or trans and wherein n is 0, 1, 2, 3 and n′ is 1, 2, or 3;
W₁ and W₂ are independently selected from CN, C≡CR, COOH, COOR wherein R is straight or branched C₁₋₄-alkyl group, CONH₂, OCH₂OCH₃ halogen;
D₁ and D₂ are independently selected from R, NO₂, halogen or O—R wherein R is a hydrogen, C₁₋₄-alkyl group optionally substituted by halogen; L is a linking group selected from (CH₂)ₐX or O(CH₂)_{b}X, (OCH₂CH₂)ₙ a and b being 0-10, n being 1-4 and X being O—C(═O)C—; and the polymer is chosen from poly(alkylacrylate)s or copolymers thereof.

27. A compound of formula (I) according to claim 26 wherein n is 0 n′ is 1; and W₁=W₂ are CN and the polymer is polymethylmethacrylate.

28. A compound of formula (I) according to claim 26 wherein n and n′ are 1, 2 or 3; D₁ and D₂ at R or OR, R being C₁-C₄alkyl optionally substituted by halogen; and W₂ are CN, COOH or CONH₂ and the polymer is methylmethacrylate.

29. A process for the preparation of a compound of formula (II) 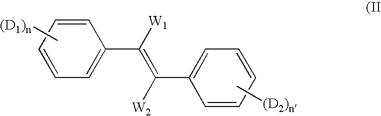
where W₁or W₂are COOH, COOR, OCH₂OCH₃, said process comprising reacting a substituted or non-substituted benzil 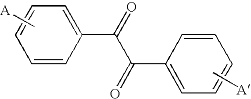
wherein A and A′ are H, halogen or OR, R being a C₁-C₄ alkyl group; with a BrCH₂C(O)OCH₂CH₃ to yield a compound of formula (IV) which may further be reacted to yield a compound of formula (V): 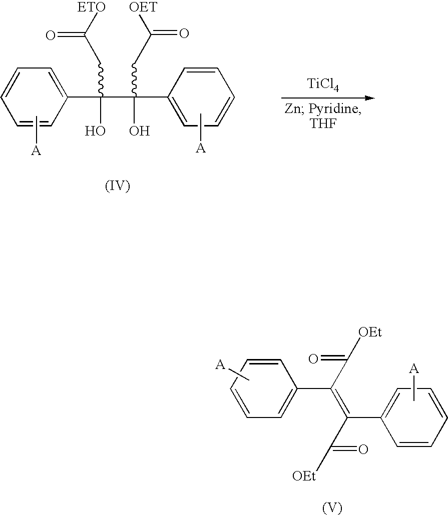
wherein compounds of formula (V) may further be reduced to yield a compound of formula (VI), which can further be reacted to yield a compound of formula (VII):

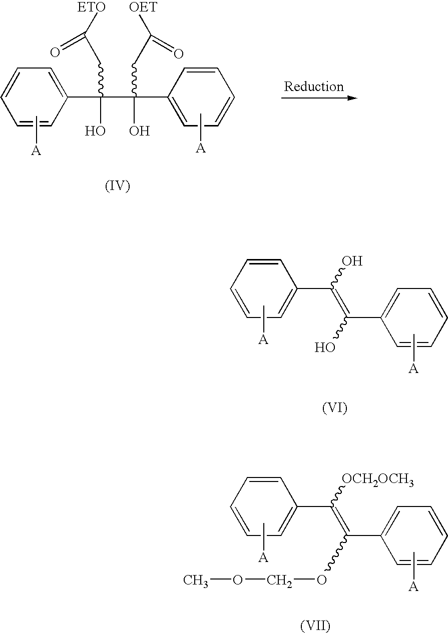

30. A process for the preparation of a compound of formula (X), said process comprises reacting a benzoylcyanide under basic conditions.

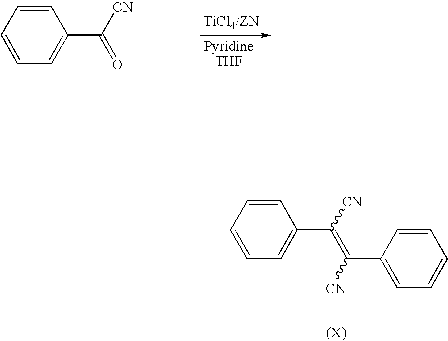

31. A process for the preparation of a compound of formula (XIV), said process comprising
(a) reacting a compound of formula XI, to form a compound of formula (XII): 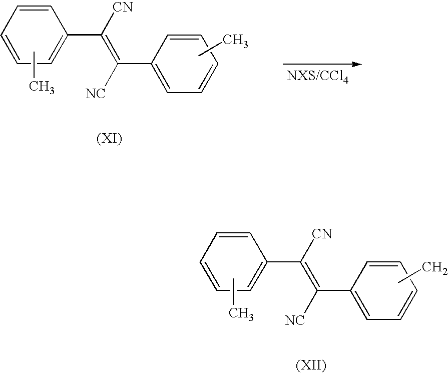
wherein X is halogen;
(b) reacting a compound of formula (XII) with a bifunctional spacer: 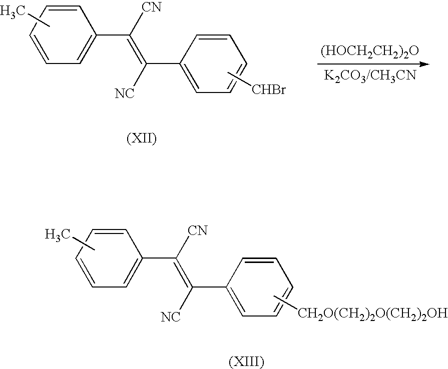
(c) transesterifying the compound of formula (XIII) with a polymer:

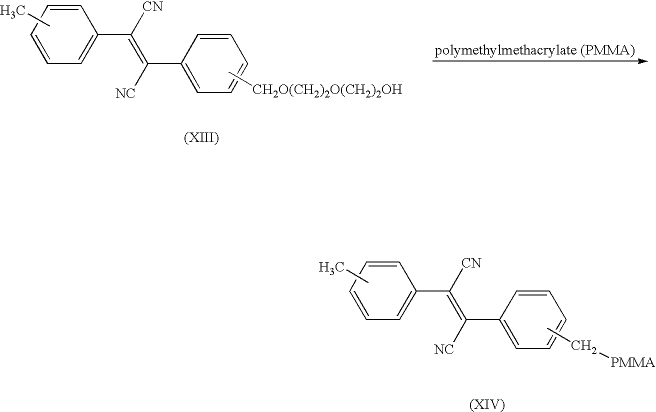

32. A process for the preparation of a copolymer of formula (XVIII), said process comprising:
(a) reacting a compound of formula (II) to yield a compound of formula (XV): 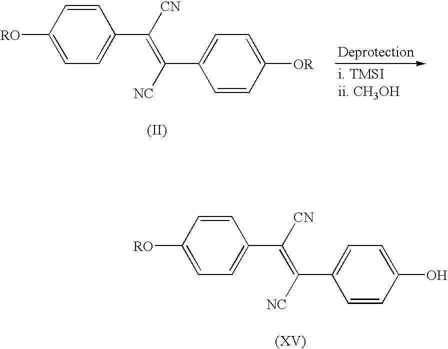
wherein R is a C₁₋₄-alkyl
(b) preparing a bi-functional spacer of formula (XVI): 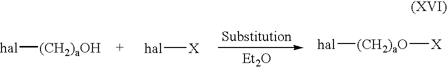
wherein a and X are as defined in claim 26;
(c) reacting said bi-functional spacer of formula (XVI) with the compound of formula (XV) to yield a compound of formula (XVII): 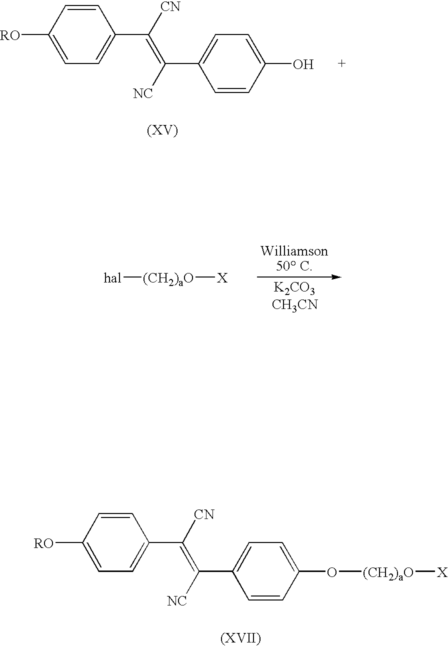
(d) polymerizing the compound of formula (XVII) in the presence of a monomer to yield a copolymer of formula (XVIII):

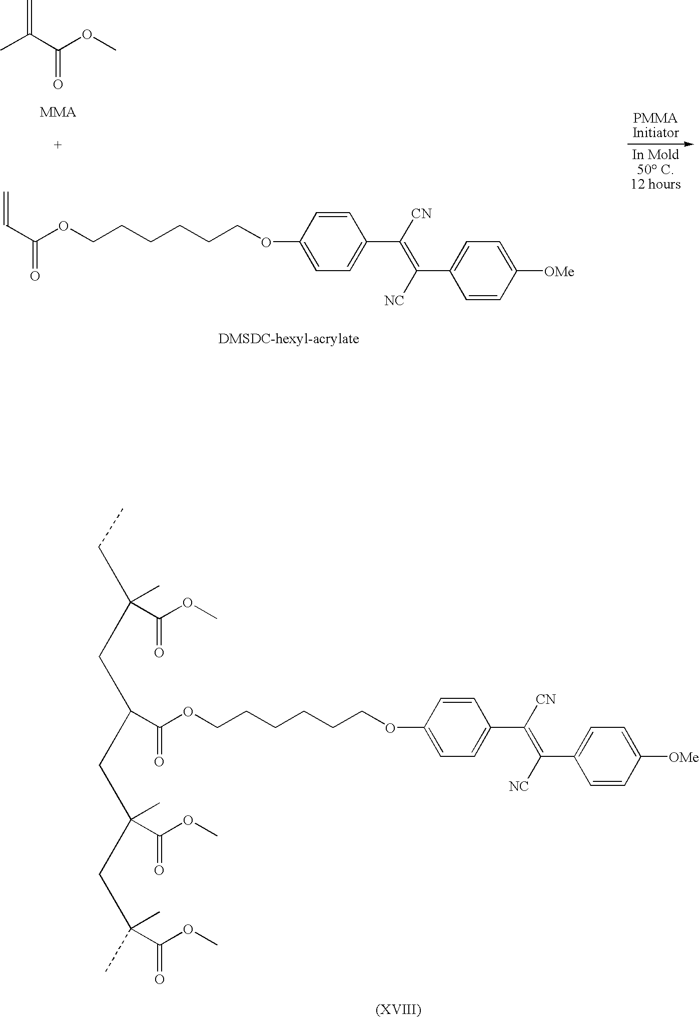

33. A process according to claims 31 or 32, wherein the polymerization step comprises the addition of at least one plasticizer.

34. A compound of formula (XVII): 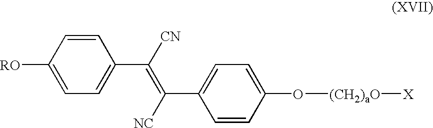
wherein R is a C₁₋₄-alkyl group, a is 1 to 10 and X is C(═O)CH═CH₂.

35. A compound of formula (XIII)

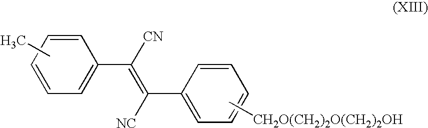

36. A copolymer of formula (XVIII).

37. A three-dimensional memory apparatus for storing information in a volume comprising an active medium, which is capable of changing predominantly from a first to a second isomeric form as a response to irradiation of a light beam having an energy substantially equal to a first excitation energy, wherein the concentration ratio between a first and a second isomeric form in any given volume portion represents a data unit; said memory apparatus being characterized in that said active medium comprises a compound of formula (II) 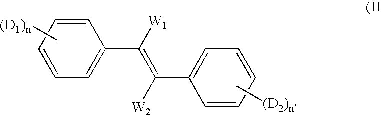
bound to a polymer according to claim 26.

38. A three dimensional memory apparatus of claim 37, wherein said compound of formula (II) is a donor-acceptor-donor compound.

39. A memory apparatus according to claim 37, comprising:
(b) means for directing a light beam having a first energy, different from said first excitation energy, to a selected portion of the active medium; and
(c) means for directing at least one additional light beam having at least one additional energy, also different from said first excitation energy, to said selected portion of the active medium;
wherein the combined energies of the first light beam and that of the at least one additional light beam are substantially equal to the first excitation energy.

40. The apparatus according to any of claims 37 to 39 further comprising means for reading the data units from the concentration ratio of the isomeric states of the active medium in different portions of said active medium.

41. The apparatus according to claim 37, wherein the two isomeric forms have a substantially different absorption coefficient for absorbing energy of second threshold energy.

42. The apparatus according to claim 41, wherein said substantially different absorption coefficient is in the infrared region.

43. An apparatus according to claim 39, wherein said means for reading the data units comprises
means for directing a first light beam having an energy different than said second excitation energy to a selected portion of the active medium; and
means for directing at least one additional light beam having at least one additional energy different than said second excitation energy, to said selected portion of the active medium;
wherein the combined energy of the first light beam and said at least one additional light beam is equal to said second excitation energy.
